(19) United States
(12) Patent Application Publication
(10) Pub. No.: US 2022/0048682 A1
(43) Pub. Date: Feb. 17, 2022

(54) SEALING ARRANGEMENT OF A FRAGRANCE CONTAINER AND FRAGRANCE CONTAINER

(71) Applicant: MAHLE International GmbH, Stuttgart (DE)

(72) Inventors: Thomas FEITH, Pforzheim (DE); Andre BAUER, Wendlingen (DE); Christian SCHEURER, Leinfelden-Echterdingen (DE)

(73) Assignee: MAHLE International GmbH, Stuttgart (DE)

(21) Appl. No.: 17/401,949

(22) Filed: Aug. 13, 2021

(30) Foreign Application Priority Data
Aug. 13, 2020 (DE) 10 2020 210 309.7

(51) Int. Cl. B65D 43/02

(52) U.S. Cl. CPC B65D 43/0202; B65D 2543/00444; B65D 2543/00296

(57) ABSTRACT

The invention relates to a sealing arrangement of a fragrance container, wherein the fragrance container has a container body and a container lid, wherein the container lid can be placed on the container body in a sealable manner, wherein the container lid has an opening in which a displaceably guided sealing lid is accommodated, wherein the container lid has at least one projecting, rigid lip running around the opening, which lip projects towards the sealing lid, wherein a flat seal is arranged on the sealing lid against which seal the at least one lip rests in the closed state of the opening. The invention also relates to a fragrance container.

This nonprovisional application claims priority under 35 U.S.C. § 119(a) to German Patent Application No. 10 2020 210 309.7, which was filed in Germany on Aug. 13, 2020, and which is herein incorporated by reference.

### BACKGROUND OF THE INVENTION

### Field of the Invention

The present invention relates to a sealing arrangement of a fragrance container, in particular of a fragrancing device of a motor vehicle, and to a fragrance container.

### Description of the Background Art

A variety of fragrancing devices are known in the state of the art. For example, passive fragrancing devices are known which release fragrance into the surrounding air on their own, wherein the release of the fragrance is not controlled in the case of passive fragrancing devices.

Fragrancing devices have also become known in which the delivery of fragrance to an air flow is controlled. Typically, the fragrancing device delivers the fragrance to an air flow in an air duct in a metered manner, wherein the amount of fragrance delivered is determined and delivered in a controlled manner in order to meter the fragrance intensity.

In order to be able to deliver the fragrance stored in a fragrance container to an air flow in a defined controlled manner, there is, for example, the possibility of conveying the fragrance from the fragrance container into the air, such as spraying it into the air. It is also possible to let the air flow to be fragranced flow through the fragrance container, so that the air flow picks up the fragrance in the process and discharges it from the fragrance container. For this purpose, the fragrance container is opened and closed in a controlled manner, wherein the fragrance container must be sealed in the closed state so that fragrance is not discharged unintentionally and thus unintentional fragrancing takes place. Problems repeatedly arise here because the seal cannot be permanently guaranteed.

### SUMMARY OF THE INVENTION

It is therefore an object of the present invention to provide a sealing arrangement by means of which a fragrance container can be sealed permanently, securely and inexpensively. It is also the object to create an improved fragrance container with secure and durable and cost-effective sealability.

An exemplary embodiment of the invention relates to a sealing arrangement of a fragrance container, wherein the fragrance container comprises a container body and a container lid, wherein the container lid can be sealingly placed on the container body, wherein the container lid comprises an opening in which a displaceably guided sealing lid is accommodated, wherein the container lid has, running around the opening, at least one projecting, rigid lip which projects towards the sealing lid, wherein a flat seal is arranged on the sealing lid, against which seal the at least one lip rests in the closed state of the opening. This ensures that the seal can be produced simply and inexpensively and that the at least one lip, resting upright against the seal, can seal the opening securely and permanently.

It is also useful if the container lid has a guide for guiding the sealing lid between a closed position, in which the sealing lid closes the opening, and an open position, in which the sealing lid releases the opening. This advantageously achieves reliable and repeatable displaceability of the sealing lid.

It is also expedient if a power storage is provided, by means of which the sealing lid can be acted upon in the direction of the container lid and the at least one lip. In this way, it can be achieved that the fragrance container is closed unactuated and does not allow any fragrance to escape.

It is also expedient if the container lid with the at least one lip and/or the sealing lid is made of an essentially dimensionally stable, hard plastic material, in particular polyamide (PA), polypropylene (PP), polyphenylene sulfide (PPS), etc. This ensures that the container lid, the lip or lips and/or the sealing lid itself remain dimensionally stable and retain their shape over time. This has advantages in conjunction with the elastic seal, as the sealability is also maintained in the long term.

It is also particularly advantageous if the seal is made of an elastic material, in particular rubber, elastomer, silicone, etc. This achieves sealability in cooperation with a hard lip.

It is also expedient if the seal is placed on the sealing lid and/or is attached to the sealing lid in a form-fitting and/or material-fitting manner and/or is injection- molded onto the sealing lid. In this way, a simply formed flat seal can be achieved which yields and seals locally when the at least one lip is in contact.

Likewise, it is advantageous if the container lid has two circumferential, rigid lips arranged concentrically to one another. This makes it possible to achieve a simple design of the opening while ensuring sealability.

It is also advantageous if, radially between the two circumferential lips, the opening is formed as an annular opening. This allows for simple arrangement and mounting of the sealing lid.

An exemplary embodiment of the invention relates to a fragrance container comprising a container body and comprising a container lid, wherein the container lid can be placed on the container body in a sealable manner, wherein the container lid has an opening in which a displaceably guided sealing lid is accommodated, wherein a sealing arrangement according to the invention is provided for sealing the opening.

It is also advantageous if the container lid can be placed on the container body and screwed or latched, wherein a sealant is arranged between the container body and the container lid. This reliably seals the connection between the container body and the container lid.

Further scope of applicability of the present invention will become apparent from the detailed description given hereinafter. However, it should be understood that the detailed description and specific examples, while indicating preferred embodiments of the invention, are given by way of illustration only, since various changes, combinations, and modifications within the spirit and scope of the invention will become apparent to those skilled in the art from this detailed description.

### BRIEF DESCRIPTION OF THE DRAWING

The present invention will become more fully understood from the detailed description given hereinbelow and the accompanying drawing which are given by way of illustration only, and thus, are not limitive of the present invention, and wherein the sole figure shows a schematic representation of a fragrance container according to the invention with a sealing arrangement according to the invention.

### DETAILED DESCRIPTION

The figure shows a schematic representation of a fragrance container 1, such as can be used in a fragrancing device in a motor vehicle.

Such an exemplary air fragrancing device may include a first air duct comprising an air inlet opening and an air outlet opening. Thus, air may enter the first air duct through the air inlet opening and the air may be discharged through the air outlet opening. A blower device may also be provided to control the flow, which sucks or blows air into the first air duct through the air inlet opening and conveys it towards the air outlet opening. The blower may be a spiral blower.

Also, by way of example, at least one second air duct may be provided which branches off from the first air duct at a first duct branch and rejoins the first air duct at a second duct branch.

Accordingly, a first fragrance container 1 can be provided in the second air duct between the first duct branch and the second duct branch, for example, through which the air flow in the second air duct can flow. As a result, a certain amount of the fragrance 2 can be delivered from the fragrance container 1 to the air flowing through the fragrance container 1, so that the air picks up the fragrance 2 and carries it away for fragrancing a vehicle interior of a motor vehicle.

For controlling the flow through the fragrance container 1 and thus also the delivery of the fragrance 2 to the air flowing through, by way of example a first valve device is provided upstream and/or downstream of the first fragrance container 1.

The fragrancing device can basically be designed as an autonomous device, which is not designed as an integrated component of the motor vehicle, but which can be operated and arranged independently. There can be an independent power supply by means of batteries or rechargeable batteries, or an electrical feed connection to the vehicle electrical system can be provided.

Alternatively, the fragrancing device could also be part of the vehicle equipment, for example part of an air conditioning system.

The figure shows a fragrance container 1 in section. The fragrance container 1 has a container body 3 and a container lid 4, wherein the container body 3 can be approximately pot-shaped. It can also be designed in another way.

The container lid 4 can be placed on the container body 3 in a sealable manner. The container lid 4 can be placed on the container body 3 and can be screwed or latched or connected in some other way, in particular also by adhesive bonding, etc. In this case, a seal 5 is preferably arranged between the container body 3 and the container lid 4 in order to seal the connection between the container body 3 and the container lid 4.

The container lid 4 has at least one opening 6, which is used for the inflow and/or outflow of air for discharging the fragrance 2.

To control the release of the opening 6, a displaceably guided sealing lid 7 is provided, which can be displaced between a closed position and an open position. In the closed position of the sealing lid 7, the at least one opening 6 is in the closed state and in the open position of the sealing lid 7, the at least one opening is in the released state.

The sealing lid 7 is displaceably guided in the opening 6. In this context, “in the opening” with regard to an opening 6 means that the sealing lid 7 engages through the opening 6 with a shaft 8, for example. In the case of a plurality of openings 6, “in the opening” would mean, for example, that the shaft 8 would be arranged between the openings 6, or arranged in one of the openings 6 or in the vicinity thereof.

A sealing arrangement 10 is provided for sealing the at least one opening 6.

The sealing arrangement 10 provides that the container lid 4 has at least one projecting, rigid lip 11 around the opening 6, which projects towards the sealing lid 7, wherein a flat seal 12 is arranged on the sealing lid 7 against which the at least one lip 11 rests in the closed state of the opening 6.

A guide 9 is provided for guiding the displaceability of the sealing lid 7, for guiding the sealing lid 7 between a closed position in which the sealing lid 7 closes the opening 6, and an open position, in which the sealing lid 7 releases the opening 6. In the exemplary embodiment shown, the guide 9 is designed as a sleeve through which the shaft 8 of the sealing lid 7 engages.

In an advantageous design, it is also advantageous if a power storage 13 is provided, by means of which the sealing lid 7 can be acted upon in the direction of the container lid 4 and the at least one lip 11. In this case, the power storage 13 is supported on the sleeve designed as a guide 9 and on the shaft 8 of the sealing lid 7.

According to an idea of the inventors, the container lid 4 with the at least one lip 11 and/or the sealing lid 7 is made of a substantially dimensionally stable, hard plastic material, in particular of polyamide (PA), polypropylene (PP), polyphenylene sulfide (PPS), etc. This hard plastic material has a higher flexural rigidity than the material of the seal 12, in particular it has a substantially higher flexural rigidity than the material of the seal 12.

Advantageously, the seal 12 is made of an elastic material, in particular rubber, elastomer, silicone, etc.

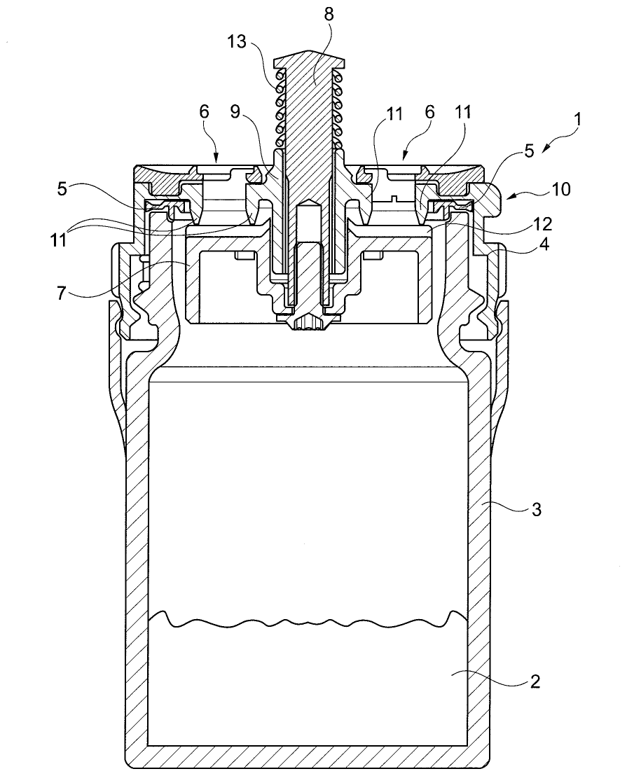

FIG. 1 shows that the seal 12 is placed on the sealing lid 7 and/or is attached to the sealing lid in a form-fitting and/or material-fitting manner and/or is injection-molded onto the sealing lid. For example, the seal 12 can be glued onto the sealing lid 7.

In the embodiment shown, the container lid 4 has two circumferential, rigid lips 11 arranged concentrically to one another. The opening 6 is formed radially between the two circumferential lips 11 as an annular opening.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A sealing arrangement of a fragrance container, the fragrance container comprising:
a container body;
a container lid adapted to be placed on the container body in a sealable manner;
an opening formed in the container lid in which a displaceably guided sealing lid is accommodated;
at least one projecting rigid lip provided on the container lid, the at least one projecting rigid lip running around the opening and projects towards the sealing lid; and
a flat seal arranged on the sealing lid; and
at least one lip resting against the seal in a closed state of the opening.

2. The sealing arrangement according to claim 1, wherein the container lid comprises a guide to guiding the sealing lid between a closed position, in which the sealing lid closes the opening and an open position, in which the sealing lid releases the opening.

3. The sealing arrangement according to claim 1, wherein a power storage is provided, via which the sealing lid is acted upon in the direction of the container lid and the at least one lip.

4. The sealing arrangement according to claim 1, wherein the container lid with the at least one lip and/or the sealing lid is formed of a substantially dimensionally stable, hard plastic material, in particular polyamide, polypropylene, or polyphenylene sulfide (PPS).

5. The sealing arrangement according to claim 1, wherein the seal is made of an elastic material, rubber, elastomer, or silicone.

6. The sealing arrangement according to claim 1, wherein the seal is placed on the sealing lid and/or is fastened in a form-fitting and/or material-fitting manner to the sealing lid and/or is injection-molded onto the sealing lid.

7. The sealing arrangement according to claim 1, wherein the container lid has two circumferential, rigid lips arranged concentrically to each other.

8. The sealing arrangement according to claim 7, wherein radially between the two circumferential lips the opening is formed as an annular opening.

9. A fragrance container comprising:
a container body;
a container lid that is adapted to be placed on the container body in a sealable manner, wherein the container lid has an opening in which a displaceably guided sealing lid is accommodated; and
a sealing arrangement according to claim 1 to seal the opening.

10. The fragrance container according to claim 9, wherein the container lid is adapted to be placed on the container body and is adapted to be screwed or latched, and wherein a seal is arranged between the container body and the container lid.
